(19) United States
(12) Patent Application Publication
(10) Pub. No.: US 2011/0311625 A1
(43) Pub. Date: Dec. 22, 2011

(54) Solid dosage forms of fenofibrate

(76) Inventors: Amruth Gowda Doddaveerappa, Hyderabad (IN); Rajesh Jagannath Kasunde, Hyderabad (IN); Nagaprasad Vishnubhotla, Hyderabad (IN); Sivakumaran Meenakshisunderam, Hyderabad (IN)

(21) Appl. No.: 13/134,672

(22) Filed: Jun. 13, 2011

(30) Foreign Application Priority Data
Jun. 14, 2010 (IN) 1650/CHE/2010

(51) Int. Cl. A61K 9/30; A61P 3/06; A61K 31/216

(52) U.S. Cl. 424/465; 514/543

(57) ABSTRACT

An improved solid dosage form of fenofibrate which exhibits improved dissolution properties leading to increased bioavailability of fenofibrate. A novel core-shell approach to the composition is provided as well as a process for the preparation of the improved solid dosage forms.

### RELATED APPLICATIONS

This application claims priority from Indian Application 1650/CHE/2010 filed on Jun. 14, 2010.

### FIELD OF THE INVENTION

The present invention relates to a solid dosage form comprising fenofibrate. The present invention also relates to a process for preparation of solid dosage form comprising fenofibrate.

### BACKGROUND OF THE INVENTION

Fenofibrate is a lipid regulating agent, chemically known as 2-[4-(4-chlorobenzoyl) phenoxy]-2-methyl-propanoic acid, 1-methylethyl ester and is disclosed in U.S. Pat. No. 4,058,552. Fenofibrate is currently marketed in the United States under the tradenames TRICOR®, FENOGLIDE® and TRIGLIDE® in the form of tablets and with the tradenames ANTARA® and LIPOFEN® in the form of capsules.

Bioavailability is the degree to which a drug becomes available to the target tissue after administration. Many factors can affect bioavailability including the dosage form and various properties, e.g., dissolution rate of the drug. Poor bioavailability is a significant problem encountered in the development of pharmaceutical compositions, particularly those containing an active ingredient that is poorly soluble in water. Poorly water soluble drugs, those having solubility less than about 10 mg/ml, tend to be eliminated from the gastrointestinal tract before being absorbed into the circulation.

The solubility of an active pharmaceutical ingredient influences the bioavailability of the drug. Fenofibrate is a poorly soluble drug. Due to its poor hydrosolubility, fenofibrate poses problem of low dissolution. It is also poorly absorbed in the digestive tract and consequently its bioavailability is incomplete and irregular. Clearly, there is a need for improved compositions in which the fenofibrate exhibits better dissolution properties.

There are several prior art references which discloses various attempts to improve the solubility of fenofibrate.

U.S. Pat. No. 4,800,079 and U.S. Pat. No. 4,961,890 discloses granules with controlled release of fenofibrate, each granule comprising an inert core, a layer based on fenofibrate and a protective layer, wherein the improvement comprises the layer based on fenofibrate containing the fenofibrate in the form of crystalline microparticles of dimensions not greater than 30 microns, said microparticles being included in the pores of an inert matrix soluble in water.

U.S. Pat. No. 4,895,726 discloses a composition containing a co-micronized mixture of particles of fenofibrate and a solid surfactant, wherein the mean particle size of said co-micronized mixture is less than 15 μm.

U.S. Pat. No. 5,145,684 discloses particles consisting essentially of a crystalline drug substance having a solubility in water of less than 10 mg/ml, said drug substance having a non-crosslinked surface modifier adsorbed on the surface thereof in an amount sufficient to maintain an effective average particle size of less than about 400 nm.

U.S. Pat. No. 6,027,747 discloses a solid dispersion comprising fenofibrate in a hydrophilic polymer and a surfactant prepared by co-precipitation.

U.S. Pat. No. 6,074,670, U.S. Pat. No. 6,596,317 and U.S. Pat. No. 7,037,529 discloses fenofibrate composition comprising inert hydrosoluble carrier covered with at least one layer containing a fenofibrate active ingredient in a micronized form having a size less than 20 μm, a hydrophilic polymer and, optionally, a surfactant and further processing them into a suitable dosage form.

U.S. Pat. No. 6,375,986 discloses solid dose nanoparticulate composition comprising at least one poorly soluble active agent, at least one polymeric surface stabilizer adsorbed to the surface of the drug, DOSS, and a pharmaceutically acceptable carrier, as well as any desired excipients. The patent further discloses the use of solid dose nanoparticulate composition for the preparation of suitable dosage form.

U.S. Pat. No. 6,696,084 discloses a process for the preparation of small particles or microparticles containing fenofibrate and a phospholipid surface stabilizing substance comprising the steps of: a) mixing at high shear an admixture of fenofibrate and a phospholipid in an aqueous carrier in the absence of an organic solvent within a temperature range at or above the melting point of the fenofibrate to form a heated suspension containing the fenofibrate, then b) homogenizing said heated suspension in a pressure range and within said temperature range to form a heated homogenate containing the fenofibrate then c) spray drying the heated homogenate to form dried small particles.

U.S. Pat. No. 6,180,138 discloses a process for preparing a dosage form comprising the steps of premixing the lipid-regulating agent with an excipient, micronizing the powdered mixture, suspending the micronized powdered mixture in a surfactant solution, drying the mixture, wet or dry granulating the mixture, and optionally forming a finished oral dosage form of the resulting formulation.

U.S. Pat. No. 6,368,622 discloses a process for preparing a solid formulation comprising the steps of forming a mixture of the lipid-regulating agent with a solid surfactant, and granulating the mixture by melting, mixing, and congealing, then optionally forming a finished dosage form.

U.S. Pat. No. 6,383,517 discloses a process for preparing a solid formulation comprising the steps of dissolving the lipid-regulating agent in a surfactant solution, premixing an excipient, wet granulating the lipid-regulating agent/surfactant solution and the premix, drying the resulting mixture, and optionally sizing the dried granules and forming a finished dosage form.

U.S. Pat. No. 6,444,225 discloses a process of making a composition comprising a solid dispersion of a disintegrant dispersed in fenofibrate, which comprises the steps of melting the fenofibrate, blending the disintegrant into the molten fenofibrate, and solidifying the mixture.

U.S. Pat. No. 6,465,011 discloses a solid formulation comprising the lipid-regulating agent dispersed in a hydrophilic, amorphous polymer in which said lipid-regulating agent is present as a metastable, amorphous phase.

U.S. Pat. No. 6,531,158 discloses a drug delivery system comprising micronized fenofibrate and an inert substrate of suitable particle size, selected from microcrystalline cellulose or lactose, which when orally administered as a single 67 mg dose in adults maintains post ingestion blood plasma levels of fenofibric acid of: at least about 100 mg/ml at one hour; at least about 350 mg/ml at two hours; at least about 750 mg/ml at four hours; at least about 850 mg/ml at five hours; and at least about 650 mg/ml at twenty-four hours.

U.S. Pat. No. 6,555,135 discloses a composition comprising a comicronized mixture of fenofibrate, and a solid non-toxic amount of a pharmaceutically acceptable excipient having no therapeutic activity that is not a surfactant.

U.S. Pat. No. 7,101,574 discloses a pharmaceutical composition in the form of granules, wherein each granule comprises a neutral microgranule on which is a composition comprising: micronized fenofibrate, a surfactant, and a binding cellulose derivative as a solubilization adjuvant.

U.S. Pat. No. 7,255,877 discloses a method of preparing fenofibrate microparticles, comprising the steps of: (1) mixing the fenofibrate particles with (a) a natural or synthetic phospholipid and (b) at least one non-ionic, anionic, or cationic surfactant to form a mixture, prior to or during a reduction of particle size, and (2) subjecting the mixture of step (1) to size reduction by an energy input procedure selected from one or more of sonication, milling, homogenization, microfluidization, or precipitation from solution using antisolvent and solvent precipitation in the presence of the mixture to produce fenofibrate microparticles having a volume-weighted mean particle size that is about 50% smaller than particles produced without the presence of the surfactant using the same energy input procedure.

U.S. Pat. No. 7,276,249 and U.S. Pat. No. 7,320,802 discloses a composition comprising nanoparticulate fibrate, preferably fenofibrate particles have an effective average particle size of less than about 500 nm and at least one surface stabilizer adsorbed on the surface of the fibrate particles.

U.S. Pat. No. 7,927,627 discloses a nanoparticulate fibrate composition comprising: (a) particles of a fibrate or a salt thereof having an effective average particle size of less than about 2000 nm; and (b) associated with the surface thereof hypromellose, dioctyl sodium sulfosuccinate, and sodium lauryl sulfate as surface stabilizers; wherein the composition is phospholipid-free.

US 2003/0224059 a drug delivery vehicle comprising at least one pharmaceutical carrier bearing microparticle of a drug, wherein the microparticles have a mean particle size of about 100 nm to about 10 μm.

US 2004/0115264 discloses fenofibrate tablet, characterized in that it is obtained by compressing a mixture comprising: a) granules containing: 1 to 5% of a surfactant; micronized fenofibrate; and at least one solid excipient selected from starch, cellulose and derivatives thereof, with the exception of C₁₂ disaccharides, said granules being obtained by granulating the mixture with the aid of an aqueous solution of polyvinylpyrrolidone; b) crosslinked polyvinylpyrrolidone; and c) optionally flow aids or lubricants, the amount of fenofibrate being greater than 50% by weight, expressed relative to the weight of the tablet.

US 2005/0112192 and US 2006/0177512 discloses a process for preparing a drug formulation comprising the steps of: dissolving a lipid-regulating drug in a solvent free of surfactant to form a drug solution; premixing an excipient to generate an admixture; wet granulating the admixture and the drug solution to form a granulated drug admixture; and drying the granulated admixture.

US 2006/0177499 discloses a dry granulation process for preparing composition comprising co-micronised mixture of fenofibrate and solid surfactant.

US 2007/0014853 discloses a dosage form comprising a granulate, wherein the granulate comprises an active pharmaceutical ingredient having a poor water solubility intimately associated with at least one pharmaceutically acceptable sugar, and wherein when the active pharmaceutical ingredient is fenofibrate, the at least one acceptable sugar is not lactose.

US 2007/0048384 discloses a composition comprising at least one active agent, at least one pharmaceutically acceptable surfactant and at least one pharmaceutically acceptable polymer, wherein the active agent is primarily amorphous fenofibrate.

US 2007/0148245 discloses a process for making a pharmaceutical composition of a drug having low aqueous solubility, the process comprising (a) fixing the drug in a strong matrix comprising at least one at least partially amorphous sugar to obtain a sugar-drug matrix; and (b) milling the sugar-drug matrix to obtain a milled sugar-drug matrix as the pharmaceutical composition, the composition being optionally further processed into a pharmaceutical formulation.

US 2008/0050450 discloses a composition comprising fenofibrate nanoparticles having an effective average particle size of less than 2000 nm, and a particle sequestrant.

US 2008/0095838 discloses a solid pharmaceutical composition for oral administration; which comprises, within one and the same phase: at least one solid and micronized lipophilic active principle, at least one surfactant, at least one cationic polymer insoluble in water at pH greater than or equal to 5, and at least one organic or inorganic acid.

US 2009/0202649 discloses formulation comprising a dispersion containing fenofibrate and at least one surfactant, optionally combined with one or more solid organic or inorganic excipients. The patent publication also discloses formulations of fenofibrate prepared by spray drying an emulsion comprising fenofibrate, at least one hydrophilic polymer and at least one surfactant onto inert substrate cores, or optionally collecting spray dried solid to obtain a pharmaceutical composition.

US 2010/0151037 discloses a composition comprising nanoparticles of fibrate, surfactant, co-surfactant, bulking agent and water and further discloses a method for the preparation of nanoparticles of a poorly water soluble drug.

US 2010/0166857 discloses a solid dispersion comprising: a plurality of coated particles comprising inert particles with a coating, wherein the coating comprises fenofibrate dispersed in a hydrophilic polymer, and wherein the inert particles comprise nonpareils; and a plurality of granules comprising micronized fenofibrate with at least one pharmaceutically acceptable excipient.

US 2011/0020455 discloses a solid dispersion comprising an active ingredient having a low solubility in water and a powdery porous carrier impregnated with and supporting the active ingredient, wherein the porous carrier comprises a porous silicon-containing carrier having a heating loss of not more than 4% by weight at a temperature of 950° C. for 2 hours.

EP 0 793 958 B1 discloses a process for the preparation of fenofibrate preparation using fenofibrate, surface-active agents and polyvinyl pyrrolidone and optionally one or more further auxiliary or auxiliaries and using mixing and granulation and subsequent drying, characterized in that firstly fenofibrate particles are mixed with polyvinylpyrrolidone particles and crosslinked polyvinylpyrrolidone particles and optionally further auxiliary particles, and the resultant mixture is then granulated with an aqueous solution of one or more surface-active agent(s) in a proportion of at least 1.5% by weight, based on the dry granules to be produced and the granules are dried.

WO 00/16749 discloses a method for preparing novel galenic formulations for providing fenofibrate with enhanced bioavailability when it is orally absorbed, and consisting in: (a) micronizing fenofibrate; (b) granulating the fenofibrate in the presence of a liquid medium comprising a surfactant, water and water-miscible alcohol; and (c) drying the resulting granular material.

WO 01/34119 discloses a solid dispersion formulation comprising a pharmaceutical compound, a water soluble carrier, such as polyethylene glycol (PEG), and a crystallization inhibitor, such as polyvinylpyrrolidone (PVP) or hydroxypropyl methylcellulose (HPMC).

WO 2004/028506 discloses an immediate release composition comprising an inert hydro-insoluble carrier with at least one layer containing fenofibrate in a micronized form, a hydrophilic polymer and a surfactant; and optionally one or several outer phases or layers.

WO 2008/016260 discloses solid dispersion comprising an amorphous fenofibrate dispersed in a water-soluble polymer.

WO 2008/075320 discloses a process for preparing a composition comprising fenofibrate, wherein the process comprises the steps of: (i) preparing a solution comprising fenofibrate, a surfactant and a hydrophilic polymer, (ii) homogenizing the solution of step (i) with one or more solvents, (iii) spraying the homogenized solution of step (ii) over one or more inert carriers, (iv) drying the granules of step (iii) and blending with one or more pharmaceutically acceptable excipients, (v) compressing the mixture of step (iv) into tablets or filling into capsules.

WO 2008/104846 discloses composition comprising unmicronized fenofibrate or a salt thereof in admixture with one or more wetting agents and one or more pharmaceutically acceptable excipients, wherein the admixture is not co-micronized before processing.

WO 2008/104852 discloses a composition comprising fenofibrate adsorbed on a pharmaceutically acceptable adsorbent optionally, along with one or more pharmaceutically acceptable excipients.

WO 2008/110534 discloses a process for the preparation of a pharmaceutical composition containing poorly soluble drug comprising the steps of: a) dissolving the drug, or a pharmaceutically acceptable salt thereof, and at least one polymer in a suitable solvent, to form a solution; b) spraying the solution onto inert pellets; and c) drying the inert pellets to remove the solvent.

WO 2009/016608 discloses composition comprising non-micronised fenofibrate and one or more pharmaceutically acceptable vehicles comprising one or more of polyethylene glycol or derivatives thereof, poloxamer, Cremophore RH 40 and vitamin E. The patent publication further discloses composition comprising non-micronized fenofibrate and cyclodextrin.

WO 2010/033179 discloses granule for a pharmaceutical composition, comprising a core, which comprises at least one active pharmaceutical ingredient intimately associated with at least one hydrophilic polymer, wherein the active pharmaceutical ingredient has a solubility in water of less than about 1 mg/ml.

WO 2010/075065 discloses a method of making microparticles comprising: (a) dissolving, melting, or suspending at least one water-insoluble active agent in at least one fatty acid or conjugated fatty acid, surfactant, hydrophilic polymer, or combinations thereof to form a mixture, and (b) mixing the mixture of step (a) with a hydrophilic or lipophilic carrier to form microparticles.

WO 2010/081623 discloses an aqueous suspension comprising crystalline fenofibrate or fenofibric acid having an average particle size of D(50) less than 250 nm, cellulose derivative, solubilizing adjuvant and a surfactant.

WO 2010/115886 discloses an adsorbate comprising an active pharmaceutical ingredient (API) being practically insoluble in water associated with a particulate and/or porous carrier, wherein the adsorbate is prepared by using a non-polar solvent or a mixture of non-polar solvents, and wherein essentially no API is in the form of precipitates, particles or crystals.

WO 2010/146606 discloses a stable nanodispersion comprising nanoparticles having a mean size less than 300 nm dispersed in a vehicle comprising a water miscible solvent and water, said nanoparticles comprising one or more drugs having a polymer and a surfactant comprising a mixture of fatty acids or its salts and sterol or its derivatives or its salts.

IN 770/MUM/2007 discloses an immediate release composition comprising an inert core with at least one layer containing fenofibrate in non-micronized form in admixture with pharmaceutically acceptable excipients and optionally one or more layers.

IN 599/MUM/2008 discloses a composition comprising micronized fenofibrate, one or more surfactants other than dioctylsulfosuccinate along with pharmaceutically acceptable excipients.

IN 1384/MUM/2008 discloses a formulation of fenofibrate with enhanced oral bioavailability, simplicity of design and manufacture and absence of food effect. The formulation comprises fenofibrate dissolved in a lipophilic surfactant, with a hydrophilic surfactant optionally added.

IN 1820/DEL/2009 discloses a fenofibrate composition in the form of capsules or tablets, comprising fenofibrate, surfactant, hydrophilic polymer and anti-foaming agents.

IN 1888/CHE/2009 discloses a pharmaceutical composition comprising a high drug load of fenofibrate, wherein the fenofibrate is present in more than 70% by weight of total composition and further discloses a process for preparing fenofibrate composition using extrusion process.

The above prior art references disclose various approaches to improve the solubility as well as bioavailability of fenofibrate. Still, there exists a need for improved formulations in which the fenofibrate exhibits better dissolution properties. The inventors of the present invention have developed a solid dosage form comprising fenofibrate which increases the rate of dissolution of fenofibrate as well as its bioavailability.

### OBJECTIVE OF THE INVENTION

The main objective of the present invention is to provide a solid dosage form comprising fenofibrate and one or more pharmaceutically acceptable excipients.

Another objective of the present invention is to provide a process for the preparation of solid dosage form comprising fenofibrate having better dissolution properties, content uniformity and equivalent bioavailability w.r.t commercialized fenofibrate dosage form.

### SUMMARY OF THE INVENTION

The present invention relates to a solid dosage form comprising:
a) tablet core comprising one or more pharmaceutically acceptable excipients,
b) a layer surrounding the tablet core comprising fenofibrate and one or more pharmaceutically acceptable excipients, and
c) optionally a film coating.

The present invention further relates to a solid dosage form comprising:
a) tablet core comprising one or more pharmaceutically acceptable excipients,
b) a layer surrounding the tablet core comprising fenofibrate, hydrophilic polymer/s, a hydrophilic carrier, optionally one or more surfactants and one or more pharmaceutically acceptable excipients, and
c) optionally a film coating.

The present invention further relates to a solid dosage form comprising fenofibrate prepared by the process comprising the steps of
a) preparing a tablet core,
b) preparing dispersion of fenofibrate in a suitable solvent,
c) coating the tablet core with fenofibrate dispersion, and
d) optionally film coating the coated tablet.

### DETAILED DESCRIPTION OF THE INVENTION

The present invention relates to a solid dosage form comprising fenofibrate and one or more pharmaceutically acceptable excipients.

The present invention relates to a solid dosage form comprising:
a) tablet core comprising one or more pharmaceutically acceptable excipients,
b) a layer surrounding the tablet core comprising fenofibrate and one or more pharmaceutically acceptable excipients, and
c) optionally a film coating.

The present invention further relates to a solid dosage form comprising:
a) tablet core comprising one or more pharmaceutically acceptable excipients,
b) a layer surrounding the tablet core comprising fenofibrate, hydrophilic polymer and one or more pharmaceutically acceptable excipients, and
c) optionally a film coating.

The present invention further relates to a solid dosage form comprising:
a) tablet core comprising one or more pharmaceutically acceptable excipients,
b) a layer surrounding the tablet core comprising fenofibrate, hydrophilic polymer, a hydrophilic carrier, optionally one or more surfactants and one or more pharmaceutically acceptable excipients, and
c) optionally a film coating.

The present invention further relates to a solid dosage form comprising:
a) tablet core comprising one or more pharmaceutically acceptable excipients,
b) a layer surrounding the tablet core comprising fenofibrate, hydrophilic polymer, a hydrophilic carrier, surfactants and one or more pharmaceutically acceptable excipients, and
c) a film coating.

“Tablet core” according to the present invention may be an inert tablet core comprising one or more pharmaceutically acceptable excipients. The tablet core may further contain fenofibrate along with one or more pharmaceutically acceptable excipients.

“Fenofibrate” according to the present invention includes, but not limited to, fenofibrate free base, its pharmaceutical acceptable salts, esters, ethers, solvates, hydrates, polymorphs and the like. Fenofibrate may be used in the range of 1-70% by weight of the composition.

“Pharmaceutically acceptable excipient/s” are the components added to pharmaceutical formulation to facilitate manufacture, enhance stability, control release, enhance product characteristics, enhance bioavailability, enhance patient acceptability, etc. Pharmaceutically acceptable excipients includes, but not limited to, diluents/fillers, binders, disintegrants, sugars, lubricants, glidants, compression aids, colors, sweeteners, preservatives, surfactants, phospholipids, suspending agents, dispersing agents, film formers, flavors, printing inks, etc.

Binders hold the ingredients in the composition together. Exemplary binders include, but not limited to, cellulose and its derivatives including, ethyl cellulose, hydroxypropyl cellulose, hydroxypropyl methylcellulose, methylcellulose and hydroxyethyl cellulose, carboxymethyl cellulose; starch and its derivatives; hydrocolloids; sugars; polyvinyl pyrrolidone and combinations comprising one or more of the foregoing binders. The binder may be used in the range of 1-15% by weight of the composition.

Diluents increase the bulk of the composition. Diluents according to the present invention include, but not limited to, sugars such as lactose, sucrose, dextrose; sugar alcohols such as mannitol, sorbitol, xylitol, lactitol; Starlac® (co-processed mixture of Starch and lactose), Microcelac® (co-processed mixture of microcrystalline cellulose and lactose), starch, modified starches, pregelatinized starch, dibasic calcium phosphate, tribasic calcium phosphate, powdered cellulose, microcrystalline cellulose, silicified microcrystalline cellulose and the like or combinations thereof. The diluent may be used in the range of 5-80% by weight of the composition.

Disintegrants according to the present invention include, but not limited to, water swellable substances, for example, cellulose and its derivatives including low-substituted hydroxypropyl cellulose; cross-linked polyvinylpyrrolidone; cross-linked sodium carboxymethylcellulose, sodium carboxy methylcellulose, microcrystalline cellulose; sodium starch glycolate; ion-exchange resins; starch and modified starches including pregelatinized starch; formalin-casein; and combinations comprising one or more of the foregoing water swellable substances. The disintegrant may be used in the range of 1-20% by weight of the composition.

Lubricants and glidants aids in the processing of powder materials. Exemplary lubricants include, but not limited to, calcium stearate, glycerol behenate, magnesium stearate, mineral oil, polyethylene glycol, sodium stearyl fumarate, stearic acid, talc, vegetable oil, zinc stearate, and combinations comprising one or more of the foregoing lubricants. Exemplary glidants include, but not limited to, talc, silicon dioxide, cornstarch and the like. The lubricant may be used in the range of 0.1-5% by weight of the composition.

Surfactants are compounds which are capable of improving the wetting of the drug and/or enhancing the dissolution. The surfactants can be selected from hydrophilic surfactants or lipophilic surfactants or mixtures thereof. The surfactants can be anionic, nonionic, cationic, and zwitterionic surfactants. Surfactants according to the present invention include, but not limited to, polyoxyethylene alkylaryl ethers such as polyoxyethylene lauryl ether, polyoxyethylene cetyl ether, polyoxyethylene stearyl ether; polyethylene glycol fatty acid esters such as PEG monolaurate, PEG dilaurate, PEG distearate, PEG dioleate; polyoxyethylene sorbitan fatty acid ester such as polysorbate 40, polysorbate 60, polysorbate 80; sorbitan fatty acid mono esters such as sorbitan monolaurate, sorbitan monooleate, sorbitan sesquioleate, sorbitan trioleate, polyoxyethylene castor oil derivates such as polyoxyl castor oil, polyoxyl hydrogenated castor oil, sodium lauryl sulphate, monooleate, monolaurate, monopalmitate, monostearate, sodium dioctyl sulfosuccinate (DOSS), lecithin, stearylic alcohol, cetostearylic alcohol, cholesterol, polyoxyethylene ricin oil, polyoxyethylene fatty acid glycerides, poloxamer, cremophore RH 40, and the like or combinations thereof. The surfactant may be used in the range of 0.0001-10% by weight of the composition.

Phospholipids according to the present invention include, but not limited to, phosphatidylcholine, phosphatidylethanolamine, phosphatidylserine, phosphatidylinositol, phosphatidylglycerol, phosphatidic acid, lysophospholipids, sphingomyelin, egg or soybean phospholipid, lecithin or combination thereof. The phospholipid may be used in the range of 0-5% by weight of the composition.

The expression “hydrophilic polymer” in the invention should be taken to mean any high molecular weight substance (greater, for example, than 300) having sufficient affinity towards water to dissolve therein or to form a gel. Examples of such polymers include, but not limited to, polyvinylpyrrolidone, copovidone, poly(vinyl alcohol), hydroxypropyl cellulose, hydroxyethyl cellulose, hydroxymethyl cellulose, hydroxypropyl methylcellulose, gelatin and the like or combinations thereof. The hydrophilic carrier may be used in the range of 0.1-20% by weight of the composition.

“Hydrophilic carrier” according to the present invention means, but not limited to, any excipient, generally hydrophilic, pharmaceutically inert, crystalline or amorphous. Examples of such excipients are derivatives of sugars, such as lactose, saccharose, sucrose, mannitol, sorbitol, cellulose and its derivatives, inorganic salts, starch or hydrolyzed starch (maltodextrin), or the like and mixtures thereof. The hydrophilic carrier may be used in the range of 5-80% by weight of the composition.

Suitable sugars according to the present invention include, but not limited to, one or more of sucrose, glucose, fructose, galactose, maltose, isomaltose, cellobiose, melibiose, gentiobiose, lactose, sorbitol, mannitol, xylitol, lactitol and the like or combinations thereof.

The tablet core according to the present invention may be prepared by any method known in the art such as wet granulation, dry granulation or direct compression of the pharmaceutically acceptable excipients. Preferably the tablet core is prepared by direct compression.

The present invention further relates to a solid dosage form comprising fenofibrate prepared by the process comprising the steps of:
a) preparing a tablet core,
b) preparing dispersion of fenofibrate in a suitable solvent,
c) coating the tablet core with fenofibrate dispersion, and
d) optionally film coating the coated tablet.

The present invention further relates to a solid dosage form comprising fenofibrate prepared by the process comprising the steps of:
a) preparing a tablet core,
b) preparing dispersion of fenofibrate along with one or more pharmaceutically acceptable excipients in a suitable solvent,
c) coating the tablets with fenofibrate dispersion, and
d) filling the tablets in the hard gelatin capsules.

The present invention further relates to a solid dosage form comprising fenofibrate prepared by the process comprising the steps of:
a) preparing a tablet core,
b) preparing dispersion of fenofibrate along with one or more pharmaceutically acceptable excipients in a suitable solvent,
c) coating the tablets with fenofibrate dispersion, and
d) optionally film coating the coated tablet.

The present invention further relates to a solid dosage form comprising fenofibrate prepared by the process comprising the steps of:
a) blending one or more pharmaceutically acceptable excipients,
b) compressing the blend of step (a) into a tablet,
c) dispersing fenofibrate and hydrophilic polymer in a suitable solvent,
d) coating the tablets of step (b) with fenofibrate dispersion of step (c), and
e) optionally film coating the coated tablet

The present invention further relates to a solid dosage form comprising fenofibrate prepared by the process comprising the steps of:
a) blending one or more pharmaceutically acceptable excipients,
b) compressing the blend of step (a) into a tablet,
c) dispersing fenofibrate, hydrophilic polymer and a hydrophilic carrier in a suitable solvent,
d) coating the tablets of step (b) with fenofibrate dispersion of step (c), and
e) optionally film coating the coated tablet.

The present invention further relates to a solid dosage form comprising fenofibrate prepared by the process comprising the steps of:
a) blending one or more pharmaceutically acceptable excipients,
b) compressing the blend of step (a) into tablet,
c) dispersing fenofibrate and hydrophilic polymer in a suitable solvent,
d) milling the fenofibrate dispersion,
e) dissolving sugar in the dispersion of step (d),
f) coating the tablets of step (b) with fenofibrate dispersion of step (e), and
g) optionally film coating the coated tablet.

Dosage form according to the present invention can be selected from the group comprising tablets, capsules, minitablets or the like or combinations thereof.

“Suitable solvent” according to the present invention can be any solvent wherein the drug can be either dissolved or dispersed such as isopropyl alcohol, ethanol, water, acetone, methylene chloride and the like or mixtures thereof.

“Dispersion” according to the present invention can be microdispersion or nanodispersion. The dispersion can be prepared and milled by methods known in the art.

Film coating composition includes one or more polymeric carriers along with one or more pharmaceutically acceptable excipients such as plasticisers, opacifier, anti-sticking agent, colorants, sugars, pore forming agent, surfactants and the like. More particularly the film coating is Opadry.

Suitable film coating polymers according to the present invention include, but not limited to, ethyl cellulose, hydroxypropyl cellulose, hydroxypropyl methylcellulose, hydroxyethyl cellulose, polyvinylpyrrolidone, polyvinyl alcohol, polyethylene oxide and the like or combinations thereof.

Suitable plasticizers according to the present invention include, but not limited to, polyethylene glycol, acetyl triethyl citrate, acetyl tributyl citrate, triethyl citrate, acetylated monoglycerides, glycerol, triacetin, propylene glycol, dibutyl phthalate, diethyl phthalate, isopropyl phthalate, dimethyl phthalate, dactyl phthalate, dibutyl sebacate, dimethyl sebacate, castor oil, glycerol monostearate, fractionated coconut oil and the like or combinations thereof.

Suitable opacifiers according to the present invention include, but not limited to, water insoluble pigments comprising titanium dioxide, calcium carbonate, calcium sulfate, magnesium oxide, magnesium carbonate, aluminum silicate, aluminum hydroxide, talc, iron oxide and the like or combinations thereof.

Suitable colorants include water soluble dyes, water insoluble pigments and natural colorants.

Suitable anti-sticking agents used according to the present invention are selected from talc, magnesium stearate and the like or a mixture thereof.

In another embodiment of the present invention, the weight of the tablet core may be in the range of 50 mg to about 1000 mg.

In another embodiment, the particle size of fenofibrate is in the range of about 10 nm to about 200 microns, preferably in the range of about 100 nm to about 100 microns and more preferably in the range of about 100 nm to about 1 microns.

In yet another embodiment, the amount of fenofibrate used may be in the range of about 10 to about 300 mg.

In another preferred embodiment of the present invention, the solid dosage form comprises:
a) tablet core comprising 30-50% w/w of lactose, 1-20% w/w of crospovidone, 30-50% w/w of silicified microcrystalline cellulose, 0.0001-10% w/w of sodium lauryl sulphate and 0.1-5% w/w of magnesium stearate,
b) a layer surrounding the tablet core comprising 1-70% w/w of fenofibrate, 1-20% w/w of hydroxypropyl methylcellulose, 0-5% w/w of lecithin and 5-30% w/w of sucrose, and,
c) film coating over the coated tablet,
   wherein the % w/w is based on total weight of the dosage form.

In another preferred embodiment, the solid dosage form comprising fenofibrate is prepared by a process comprising the steps of:
a) blending lactose, sodium lauryl sulphate, crospovidone and silicified microcrystalline cellulose,
b) lubricating the blend of step (a) with magnesium stearate,
c) compressing the blend of step (b) into tablet,
d) dispersing fenofibrate and hydroxypropyl methylcellulose in purified water and stirring to get uniform dispersion,
e) milling the fenofibrate dispersion to get fenofibrate having an average particle size above 600 nm,
f) dissolving sucrose and/or lecithin in the dispersion of step (e),
g) coating the tablets of step (c) with fenofibrate dispersion of step (f), and
h) applying a film coating over the coated tablets of step (g).

The following examples further exemplify the invention and are not intended to limit the scope of the invention. It is obvious to those skilled in the art to find out the composition for other dosage forms and substitute the equivalent excipients as described in this specification or with the one known to the industry.

### Example 1

| S. No | Ingredients | mg/tablet |
|---|---|---|
| | Inert Tablet Core | |
| 1 | Lactose | 258.95 |
| 2 | Sodium lauryl sulfate | 10.57 |
| 3 | Crospovidone | 13.05 |
| 4 | Silicified microcrystalline cellulose | 151.65 |
| 5 | Magnesium stearate | 0.91 |
| | Drug nano-dispersion coating | |
| 6 | Fenofibrate | 145 |
| 7 | Hydroxypropyl methylcellulose | 14.5 |
| 8 | Sucrose | 145 |
| 9 | Purified water | q.s |
| | Drug loaded tablet weight | 739.5 |
| | Film coating | |
| 10 | Opadry | 15 |
| 11 | Purified water | q.s |
| | Film coated tablet weight | 754.5 |

The processing steps involved in manufacturing fenofibrate tablets are given below:
i) lactose, sodium lauryl sulphate, crospovidone and silicified microcrystalline cellulose were sifted separately and blended,
ii) the blend of step (i) was lubricated with magnesium stearate and
iii) the lubricated blend of step (ii) was compressed into tablets,
iv) fenofibrate and hydroxypropyl methylcellulose were dispersed in water and stirred to get a uniform dispersion,
v) the dispersion of step (iv) was nanonized to get fenofibrate having an average particle size above 600 nm,
vi) sucrose was added to the nanodispersion of step (v),
vii) the tablets prepared in step (iii) were coated with nanodispersion of step (vi) and dried, and
viii) the coated tablets obtained in step (vii) were coated with Opadry coating.

### Example 2

| S. No | Ingredients | mg/tablet |
|---|---|---|
| | Inert Tablet Core | |
| 1 | Lactose | 229.39 |
| 2 | Sodium lauryl sulfate | 40.00 |
| 3 | Crospovidone | 13.05 |
| 4 | Silicified microcrystalline cellulose | 151.65 |
| 5 | Magnesium stearate | 0.91 |
| | Drug nano-dispersion coating | |
| 6 | Fenofibrate | 145 |
| 7 | Hydroxypropyl methylcellulose | 14.5 |
| 8 | Sucrose | 145 |
| 9 | Purified water | q.s |
| | Drug loaded tablet weight | 739.5 |
| | Film coating | |
| 10 | Opadry | 15 |
| 11 | Purified water | q.s |
| | Film coated tablet weight | 754.5 |

### Example 3

| S. No | Ingredients | mg/tablet |
|---|---|---|
| | Inert Tablet Core | |
| 1 | Lactose | 223.10 |
| 2 | Sodium lauryl sulfate | 40.00 |
| 3 | Crospovidone | 20.00 |
| 4 | Silicified microcrystalline cellulose | 151.00 |
| 5 | Magnesium stearate | 0.90 |
| | Drug nano-dispersion coating | |
| 6 | Fenofibrate | 145 |
| 7 | Hydroxypropyl methylcellulose | 14.5 |
| 8 | Sucrose | 145 |
| 9 | Purified water | q.s |
| | Drug loaded tablet weight | 739.5 |
| | Film coating | |
| 10 | Opadry | 15 |
| 11 | Purified water | q.s |
| | Film coated tablet weight | 754.5 |

### Example 4

| S. No | Ingredients | mg/tablet |
|---|---|---|
| | Inert Tablet Core | |
| 1 | Lactose | 258.82 |
| 2 | Sodium lauryl sulphate | 10.57 |
| 3 | Crospovidone | 13.05 |
| 4 | Silicified microcrystalline cellulose | 151.65 |
| 5 | Magnesium stearate | 0.91 |
| | Drug nano-dispersion coating | |
| 6 | Fenofibrate | 145.00 |
| 7 | Hydroxypropyl methylcellulose | 14.50 |
| 8 | Sucrose | 145.00 |
| 9 | Lecithin | 1.00 |
| 10 | Purified water | q s |
| | Drug loaded tablet weight | 740.5 |
| | Film coating | |
| 11 | Instacoat | 15.50 |
| 12 | Purified water | q s |
| | Film coated tablet weight | 756.00 |

### Example 5

| S. No | Ingredients | mg/tablet |
|---|---|---|
| | Inert Tablet Core | |
| 1 | Lactose | 258.85 |
| 2 | Sodium lauryl sulfate | 10.57 |
| 3 | Crospovidone | 13.05 |
| 4 | Silicified microcrystalline cellulose | 151.65 |
| 5 | Magnesium stearate | 0.91 |
| | Drug nano-dispersion coating | |
| 6 | Fenofibrate | 145 |
| 7 | Hydroxypropyl methylcellulose | 29 |
| 8 | Sucrose | 141.81 |
| 9 | Microcrystalline cellulose | 3.19 |
| 10 | Purified water | q.s |
| | Drug loaded tablet weight | 754 |
| | Film coating | |
| 11 | Opadry | 20 |
| 12 | Purified water | q.s |
| | Film coated tablet weight | 774 |

### Example 6

| S. No | Ingredients | mg/tablet |
|---|---|---|
| | Inert Tablet Core | |
| 1 | Lactose | 258.85 |
| 2 | Sodium lauryl sulfate | 10.57 |
| 3 | Crospovidone | 13.05 |
| 4 | Silicified microcrystalline cellulose | 151.65 |
| 5 | Magnesium stearate | 0.91 |
| | Drug nano-dispersion coating | |
| 6 | Fenofibrate | 145 |
| 7 | Hydroxypropyl methylcellulose | 29 |
| 8 | Sucrose | 135.43 |
| 9 | Microcrystalline cellulose | 3.19 |
| 10 | Crospovidone | 6.38 |
| 11 | Purified water | q.s |
| | Drug loaded tablet weight | 754 |
| | Film coating | |
| 12 | Opadry | 20 |
| 13 | Purified water | q.s |
| | Film coated tablet weight | 774 |

### Example 7

| S. No | Ingredients | mg/tablet |
|---|---|---|
| | Inert Tablet Core | |
| 1 | Lactose | 258.85 |
| 2 | Sodium lauryl sulfate | 10.57 |
| 3 | Crospovidone | 13.05 |
| 4 | Silicified microcrystalline cellulose | 151.65 |
| 5 | Magnesium stearate | 0.91 |
| | Drug nano-dispersion coating | |
| 6 | Fenofibrate | 145 |
| 7 | Hydroxypropyl methylcellulose | 29 |
| 8 | Sucrose | 100.34 |
| 9 | Microcrystalline cellulose | 31.9 |
| 10 | Crospovidone | 12.76 |
| 11 | Purified water | q.s |
| | Drug loaded tablet weight | 754 |
| | Film coating | |
| 12 | Opadry | 20 |
| 13 | Purified water | q.s |
| | Film coated tablet weight | 774 |

The compositions given in Examples 2 to 7 were prepared using the similar procedure described in Example 1.

Table 1 given below shows the comparative dissolution profile of fenofibrate tablets according to the present invention (Examples 1-3) and Tricor® Tablets carried out in 1000 ml medium (water+0.05M sodium lauryl sulphate) using Apparatus USP II (Paddle), at 50 rpm speed.

TABLE 1

| | % Drug released | | | |
|---|---|---|---|---|
| Time in min | Example-1 | Example-2 | Example-3 | Tricor ® 145 mg |
| 10 | 85 | 81 | 76 | 92 |
| 30 | 93 | 95 | 97 | 96 |
| 45 | 94 | 96 | 98 | 96 |
| 60 | 96 | 96 | 98 | 96 |

We claim:

1. A solid dosage form comprising:
a) tablet core comprising one or more pharmaceutically acceptable excipients,
b) a layer surrounding the tablet core comprising fenofibrate and one or more pharmaceutically acceptable excipients, and
c) optionally a film coating.

2. The solid dosage form according to claim 1, wherein the layer further comprises hydrophilic polymer, hydrophilic carrier or the combinations thereof.

3. The solid dosage form according to claim 2, wherein the hydrophilic polymer is hydroxypropyl methyl cellulose and hydrophilic carrier is sucrose.

4. The solid dosage form according to claim 1, wherein the pharmaceutically acceptable excipient is selected from the group consisting of diluents/fillers, binders, disintegrants, lubricants, glidants, surfactants, phospholipids and film formers.

5. The solid dosage form according to claim 4, wherein the diluent can be selected from group consisting of lactose, sucrose, powdered cellulose, microcrystalline cellulose, silicified microcrystalline cellulose or combinations thereof.

6. The solid dosage form according to claim 4, wherein the disintegrant is selected from group consisting of cross-linked polyvinylpyrrolidone; cross-linked sodium carboxymethylcellulose, sodium starch glycolate or combinations thereof.

7. The solid dosage form according to claim 4, wherein the surfactant is selected from group consisting of sodium lauryl sulphate, lecithin, sodium dioctyl sulfosuccinate or combinations thereof.

8. The solid dosage form according to claim 4, wherein the binder is selected from the group consisting of hydroxypropyl methylcellulose, sugars; polyvinyl pyrrolidone and combinations thereof.

9. A solid dosage form comprising fenofibrate prepared by the process comprising the steps of:
a) preparing a tablet core,
b) preparing dispersion of fenofibrate in a suitable solvent,
c) coating the tablet core with fenofibrate dispersion, and
d) optionally film coating the coated tablet.

10. A solid dosage form comprising:
a) tablet core comprising 30-50% w/w of lactose, 1-20% w/w of crospovidone, 30-50% w/w of silicified microcrystalline cellulose, 0.0001-10% w/w of sodium lauryl sulphate and 0.1-5% w/w of magnesium stearate,
b) a layer surrounding the tablet core comprising 1-70% w/w of fenofibrate, 1-20% w/w of hydroxypropyl methylcellulose, 0-5% w/w of lecithin and 5-30% w/w of sucrose, and,
c) film coating over the coated tablet,
wherein the % w/w is based on total weight of the dosage form.
